(19) United States
(12) Patent Application Publication
(10) Pub. No.: US 2018/0268507 A1
(43) Pub. Date: Sep. 20, 2018

(54) METHODS AND SYSTEMS FOR MANAGING PERFORMANCE BASED SLEEP PATIENT CARE PROTOCOLS

(71) Applicant: General Electric Company, Schenectady, NY (US)

(72) Inventors: Christopher Donald Johnson, Niskayuna, NY (US); Kunter Seref Akbay, Niskayuna, NY (US); Daniel Joseph Cleary, Schenectady, NY (US)

(21) Appl. No.: 15/987,714

(22) Filed: May 23, 2018

Related U.S. Application Data
(63) Continuation of application No. 13/630,315, filed on Sep. 28, 2012.

(51) Int. Cl. G06Q 50/22; G16H 40/20

(52) U.S. Cl. CPC G06Q 50/22; G16H 40/20; G06F 19/3481

(57) ABSTRACT

Methods, systems and non-transitory computer readable media for automated workflow management for sleep quality are presented. A subject in a resting state is monitored using one or more sensors for acquiring one or more physiological parameters. A phase of sleep of the subject at a specific time using the physiological parameters is detected and inferred. Further, a recuperative benefit of the detected sleep phase is estimated. Additionally, it is determined is if an activity in a patient care workflow scheduled proximate to the specific time hinders sleep of the subject. The patient care workflow is then automatically reconfigured if scheduled activity hinders patient sleep, if the determined recuperative benefits exceed a designated threshold and/or if a proposed reconfiguration satisfies one or more designated criteria accounting for both restorative benefits to the patient and objectives of competing care delivery activities for both a given patient and a department.

### CROSS-REFERENCE TO RELATED APPLICATION

This application is a continuation of U.S. application Ser. No. 13/630,315, entitled “METHODS AND SYSTEMS FOR MANAGING PERFORMANCE BASED SLEEP PATIENT CARE PROTOCOLS”, filed Sep. 28, 2012, which is herein incorporated by reference in its entirety for all purposes.

### BACKGROUND

Sound sleep is generally beneficial and restorative for a person's health and exerts favorable influence on the quality of a patient's recuperative progress. The human sleep/wake cycle typically conforms to a circadian rhythm regulated by a biological clock. The sleep cycle of a healthy person, for example, may be characterized by a general decrease in metabolic rate, body temperature, blood pressure, breathing rate, heart rate, cardiac output, sympathetic nervous activity and other physiological functions. These characteristics may be observed over several stages of the sleep cycle typically including rapid eye movement (REM) and non-rapid eye movement (NREM) sleep.

Generally, different sleep phases may impart different health benefits. By way of example, while REM sleep revitalizes the mind, later stages of non-REM sleep are generally recuperative for the person's body. Together, the REM and non-REM sleep cycles aid in preserving a healthy biological rhythm that controls hormones and neurotransmitters that determine appetite, fertility and mental and physical health. Additionally, sound sleep strengthens the immune system and is significant for detoxification, recovery and regeneration of muscles, bones, nerves and other tissues in the human body.

Particularly, convalescing patients require generous amounts of sleep for expedited recovery and restoration. Care providers, however, require access to the patient for performing various clinical procedures, which typically conflict with patient sleep. Generally, disruption of sound sleep may lead to changes in physiological parameters that hamper the patient's health, stress the immune system and impede recovery from wear and tear, and illnesses. Poor sleep quality, thus may provide an indication of deteriorating health of the patient. Accordingly, sleep/wake patterns and various physiological parameters such as heart rate and respiration during different sleep phases may be monitored to provide clinical markers for identifying and treating various health conditions afflicting the patient.

To that end, certain sleep monitoring systems employ air bladders, vibration and other electronic and motion sensors to monitor the patient's movements and sleep patterns. Certain hospital-based sleep monitoring systems employ a sleep laboratory, where the patient is connected to a polysomnography (PSG) machine that records multiple physiological parameters. Use of such systems, however, may entail tethering of multiple sensors and/or cables with electrodes to the patient, often resulting in disruptive sleep patterns due to anxiety and/or physical discomfort.

Furthermore, in an actual hospital setting, conventional workflows for patient care are often fixed in advance and followed rigidly. These workflows may include specific activities such as cleaning, medication, therapy, and/or doctor and family visitations that are scheduled for designated times and often entail disturbing or waking the patient from sleep. Particularly, in scenarios where the patient is located in a ward or a room being shared with one or more other patients, uncoordinated hospital workflows may further compound sleep disruption.

Accordingly, performing scheduled or unscheduled activities at times irrespective of a sleep phase of the patient degrades recuperative benefits the person may experience from restful sleep, thus prolonging the healing process, adding to medical and operational costs and contributing to lower patient satisfaction with their care experience. However, owing to a plurality of interdependencies involved, for example, availability of mutually exclusive resources for patient care such as doctors, equipment and supporting staff, reconfiguring a workflow in real-time without hindering other patients is a challenge presently not able to be managed for lack of a systemic approach to sleep management. Given the ever-increasing pressure on hospital productivity, left unmanaged, sleep interruptions will most likely increase as fewer staff must execute care protocols, especially during evening and night shifts.

### BRIEF DESCRIPTION

Certain aspects of the present disclosure are drawn to methods, systems and non-transitory computer readable media for automated workflow management are disclosed. A subject in a resting state is monitored using one or more sensors for acquiring one or more physiological parameters. Further, a phase of sleep of the subject at a specific time is detected using the physiological parameters and a recuperative benefit of the detected sleep phase to the subject is estimated. Additionally, it is determined is if an activity in a patient care workflow scheduled proximate to the specific time hinders sleep of the subject. The patient care workflow is then automatically reconfigured if scheduled activity hinders patient sleep, if the determined recuperative benefits exceed a designated threshold, if a proposed reconfiguration satisfies one or more designated criteria, or combinations thereof.

### DRAWINGS

These and other features and aspects of embodiments of the present disclosure will become better understood when the following detailed description is read with reference to the accompanying drawings in which like characters represent like parts throughout the drawings, wherein:

FIG. 1 is a block diagram of an exemplary system for monitoring a subject in a resting state, in accordance with aspects of the present disclosure;

FIG. 2 is a block diagram of an exemplary reasoning engine for use in automatically reconfiguring a patient care workflow, in accordance with aspects of the present disclosure;

FIG. 3 is a schematic diagram illustrating an exemplary implementation of the reasoning engine for use in automatically reconfiguring a patient care workflow, in accordance with aspects of the present disclosure; and

FIG. 4 is a flow chart illustrating an exemplary method for automated workflow management for patient care, in accordance with aspects of the present disclosure.

### DETAILED DESCRIPTION

The following description presents systems and methods for automated management of patient care workflows based on a patient's state. Particularly, certain embodiments illustrated herein describe efficient methods and systems that non-intrusively monitor the patient in a resting state and may reconfigure scheduled patient care activities in real-time based on an assessed recuperative benefit associated with the resting state and an availability of the interdependent patient care resources. As used herein, the term “resting state” corresponds to a state of the subject, in which the subject typically exhibits negligible or insignificant motion, such as when the subject is sleeping, resting, relaxing or meditating. While there may be some movement that may occur during such resting state in a healthy subject, such movement is generally considered insignificant.

One technical effect of the methods and systems of the present disclosure is to schedule patient care workflow to enable a specific duration for sleep and then reconfiguring the patient care workflow in real-time based on a sleep phase of the patient. In particular, embodiments of the methods and systems are drawn to a workflow management system configured so as to non-invasively detect a phase of sleep of the patient, determine a recuperative benefit of the detected sleep phase, identify any scheduled activities or new activities that may disrupt the sleep phase, compare the recuperative benefit with the logistics for rescheduling the patient care workflow and reschedule the workflow in real-time for optimizing the recuperative benefits of sleep for the patient. The rescheduled workflow, in turn, allows for achievement of better sleep in complex environments, such as hospitals, where conflicting care priorities degrade a patient's experience and ability to rest.

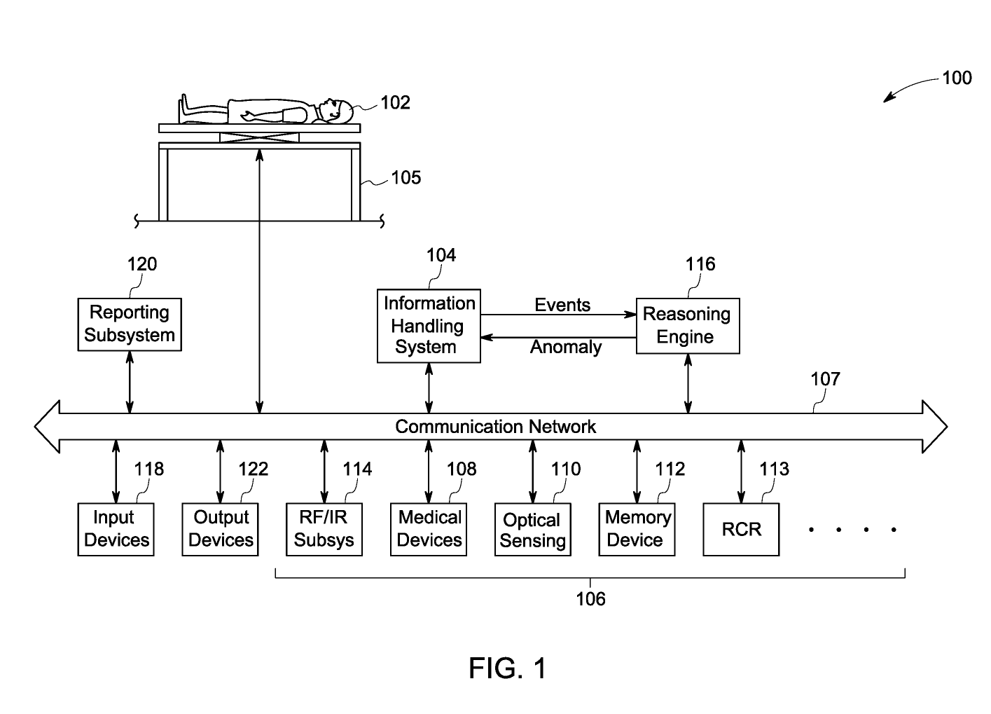

Although embodiments of the present systems and methods are discussed with reference to a hospital environment, certain embodiments of the present systems and method may also be used in an assisted healthcare, ambulatory and/or home environment for reorganizing patient care workflows in real-time to allow the patient to benefit from optimal recuperation derived from sleep. Further, embodiments of the disclosed methods and systems may also apply to veterinary practice, biofeedback applications that may employ active (for example, meditative) and/or passive feedback (for example, response feedback from stimuli such as advertising and/or other media content), certain medical protocols such as imaging and/or certain test timing control implementations. An exemplary environment that is suitable for practicing various implementations of the present systems and methods is described in the following sections with reference to FIG. 1.

FIG. 1 illustrates an exemplary workflow management system 100 for monitoring a subject such as a person or an animal (hereinafter, referred to as patient 102) in a resting state and scheduling delivery of healthcare to the patient 102 to allow for optimized recovery. Accordingly, in one embodiment, the system 100 may include an information handling subsystem 104 configured to control monitoring of the patient 102 disposed on a bed 105 and reconfigure patient care workflows that prevent disruption of patient sleep in real-time, thus allowing for faster recuperation and healing. In certain embodiments, the subsystem 104 may be configured to provide centralized control over monitoring and rescheduling operations. However, in other embodiments, the functioning of the subsystem 104 may be implemented using a plurality of distributed systems operationally coupled to the patient 102 and/or the various monitoring and patient care systems.

Although, the present embodiment describes the patient 102 disposed on the bed 105, for example, in a supine position, in alternative embodiments, the system 100 may also be configured to monitor and customize patient care workflows, for example, for the patient 102 disposed in a chair, or in other suitable positions. To that end, in one embodiment, the system 100 includes a plurality of sensors 106 disposed in a region of interest (ROI) that allows monitoring of one or more parameters corresponding to the patient 102, a caregiver and/or ambient conditions. The sensors 106, for example, may be communicatively coupled to the subsystem 104 over a communications network 107 including wired networks such as LAN and cable, and/or wireless networks such as WLAN, cellular networks, satellite networks, and/or short-range networks such as ZigBee wireless sensor networks.

Specifically, the sensors 106 may be configured for periodically or continuously monitoring the parameters that affect recovery and restoration of patient health based on a designated patient care workflow, a determined state of health of the patient 102 and/or user-defined specifications. By way of example, if health of the patient 102 is unstable, the sensors 106 may be configured to continuously monitor the ROI including the patient 102 and reconfigure the patient care workflow for maximizing rest and recovery by rescheduling certain activities such as cleaning.

Accordingly, the sensors 106 may include a plurality of devices that monitor not only the physiological parameters of the patient 102, but also ambient conditions that affect sleep quality. By way of example, in one embodiment, the sensors 106 may include one or more local, remote and/or wearable medical devices 108 configured for monitoring one or more physiological parameters such as heartbeat, respiration, pressure, volume and oxygenation of the patient blood. The medical devices 108, for example, may include magnetic resonance imaging (MRI) system, a computed tomography (CT) system, an ultrasound system, an electrocardiogram (ECG) machine, a blood pressure monitor, an X-ray machine, an oxygen monitor, an intravenous monitor and/or an anesthesia monitor.

In certain embodiments, the sensors 106 may also include an optical sensor system coupled to reasoning algorithms (hereinafter referred to as optical sensing 110), where the optical sensing 110 may be configured to identify and/or monitor one or more ROIs. The ROI, for example, may include two-dimensional (2D) and/or three-dimensional (3D) regions such as encompassing the bed 105, a wash area and/or medical devices disposed proximate to the patient 102. In one embodiment, the optical sensing 110 may be configured to identify the ROIs by detecting an identifier and/or a measured location and orientation of optical markers disposed in the patient's room. The optical markers, for example, may include one or more of graphic symbols, reflective material and patterns of unique identification and/or orientation that may be detected and tracked using the optical sensing 110.

In an alternative embodiment, however, the optical sensing 110 may be configured to identify the ROIs and one or more resources of interest based on association of predetermined shapes, patterns, color contrasts and/or reflections stored in a memory device 112 operationally coupled to the sensors 106 and/or the subsystem 104. To that end, the memory device, for example, may include a random access memory, a read-only memory, a disc drive, a solid-state memory device, and/or a flash memory, configured to store the predetermined information, scheduled patient care workflows and/or monitoring information acquired by the optical sensing 110.

Additionally, the optical sensing 110 may also be configured to detect motion in designated ROIs for determining protocol adherence by caregivers, movement of resources in and out of the patient room and/or monitoring physiological parameters of the patient 102. Accordingly, in certain embodiments, more than one optical sensing device may be disposed at different positions and orientations in the patient room, such as on the ceiling or a sidewall for allowing monitoring of multiple ROIs. In certain other embodiments, the sensors 106 may include one or more range-controlled radars 113 configured for non-invasively monitoring one or more of activity levels and physiological parameters such as heartbeat and/or respiration of the patient 102. Particularly, in scenarios where the person is located in a ward or a room being shared with one or more other patients, the range-controlled radars may allow selective focus on one or more of the occupants of a shared space. To that end, the range-controlled radar may be coupled to a directional antenna for constraining the radar signal over the desired portions of the shared space.

In further embodiments, the sensors 106 may also include a radio frequency (RF) and/or infrared (IR) subsystem 114, hereinafter referred to as RF/IR subsystem 114. The RF/IR subsystem 114 may further include one or more transmitters coupled in communication with one or more receivers and/or tags to track location and movement of resources such as staff and medical deliverables that may enter into or leave from the area of interest. Similarly, the RF/IR subsystem 114 may include IR technology that may be employed independently of, or in combination with, the RFID technology, for example, to track movement of tags coupled to resources of interest.

Although, FIG. 1 illustrates certain exemplary sensing devices, in certain embodiments, the sensors 106 may include fewer or greater number of devices. By way of example, in a hospital-based sleep laboratory, the sensors 106 may include additional devices such as accelerometers, voice recognition systems, and electromagnetic transmitters and receivers for identifying and monitoring physiological data, ambient information and/or movement of resources in and out of the patient room. In a home setting, however, the sensors 106 may include only a range-control radar system 113 configured to monitor the patient 102, generate signals indicative of the physiological parameters of the patient 102, estimate patient sleep quality using the physiological parameters and reconfigure the scheduled patient care workflow based on the estimated sleep quality.

Generally, the system 100 allows for patient care workflow reconfiguration based on the monitoring information. To that end, the system 100 may include a reasoning engine 116 configured to receive and analyze the monitoring information for providing feasible reconfiguration decisions. In certain embodiments, the system 100 queries the reasoning engine 116 at designated intervals for seeking reconfiguration decisions. In certain other embodiments, the system 100 directs the reasoning engine 116 to provide reconfiguration decisions when the patient 102 is determined to be in a specific phase of sleep. In further embodiments, the system 100 allows a user to specify input for eliciting reconfiguration decisions. To that end, in one embodiment, the system 100 may include one or more input devices 118, such as a graphical user interface (GUI), to receive user input. In certain embodiments, the user input may be used to configure one or more of the sensors 106 for focusing over a desired ROI, specifying a duration and frequency of monitoring and/or selecting specific sensing data for reporting and prompting the reasoning engine 116 to initiate evaluation for workflow reconfiguration in real time and/or offline mode.

In one embodiment, reasoning engine 116 may provide the reconfiguration decisions depending upon a phase of sleep, medical condition and/or a state of recovery of the patient 102. In certain embodiments, in addition to the patient state, the reasoning engine 116 may consider a nature of scheduled activities, projected availability of resources, recuperative benefits expected from the reconfiguration and/or user specifications for arriving at the reconfiguration decisions. The scheduled activities, in one example, may include medical examinations, medication, therapy, nutrition, diagnosis, housekeeping, visitations and/or ambient environment control.

If analysis of the monitoring information indicates presence of a mandatory activity in the scheduled workflow, unavailability of resources at a later time and/or marginal recuperative benefits, the reasoning engine 116 may communicate non-feasibility of workflow reconfiguration to the subsystem 104. Alternatively, if reconfiguring the patient care workflow is determined to be beneficial, the reasoning engine 116 may communicate the reconfigured patient care workflow to the memory device 112, the subsystem 104 and/or a reporting subsystem 120 operationally coupled to the subsystem 104. The subsystem 104, in turn, may communicate the proposed workflow changes to relevant care-providers and/or healthcare systems through the reporting subsystem 120.

To that end, the reporting subsystem 120 may include one or more output devices 122 configured to generate a designated signal, tactile output, an audio output and/or, a visual output for notifying appropriate care providers and/or associated healthcare systems of a change in patient care schedule. The output devices 122, for example, may include a whiteboard, one or more computer terminals, flat panel screens, speakers, pagers and/or suitable mobile devices. The reporting subsystem 120 may use the output devices 122 for displaying a message, sounding an alarm, sending a voicemail, text message and/or email to a mobile device of appropriate healthcare personnel, the associated healthcare systems and/or to another monitoring system through a wired and/or wireless link. In certain embodiments, the reporting subsystem 120 may customize the alerts to include indications such as use of different colors, sounds, shapes and/or patterns for highlighting processes, related departments, resources, care providers and patients affected by the workflow reconfiguration.

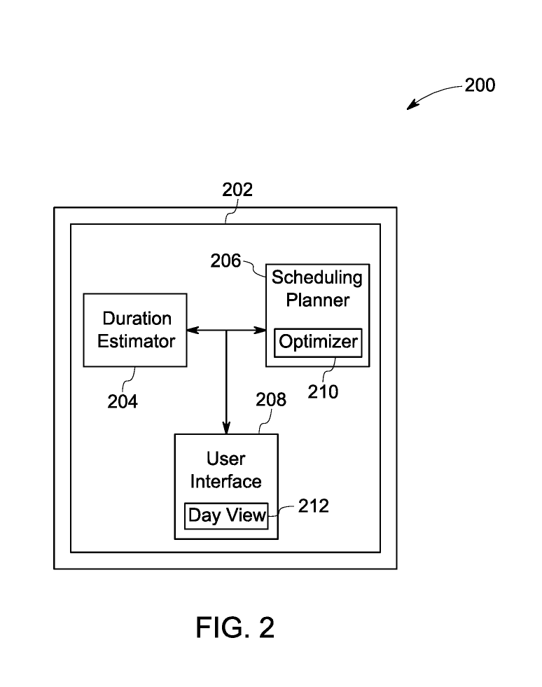

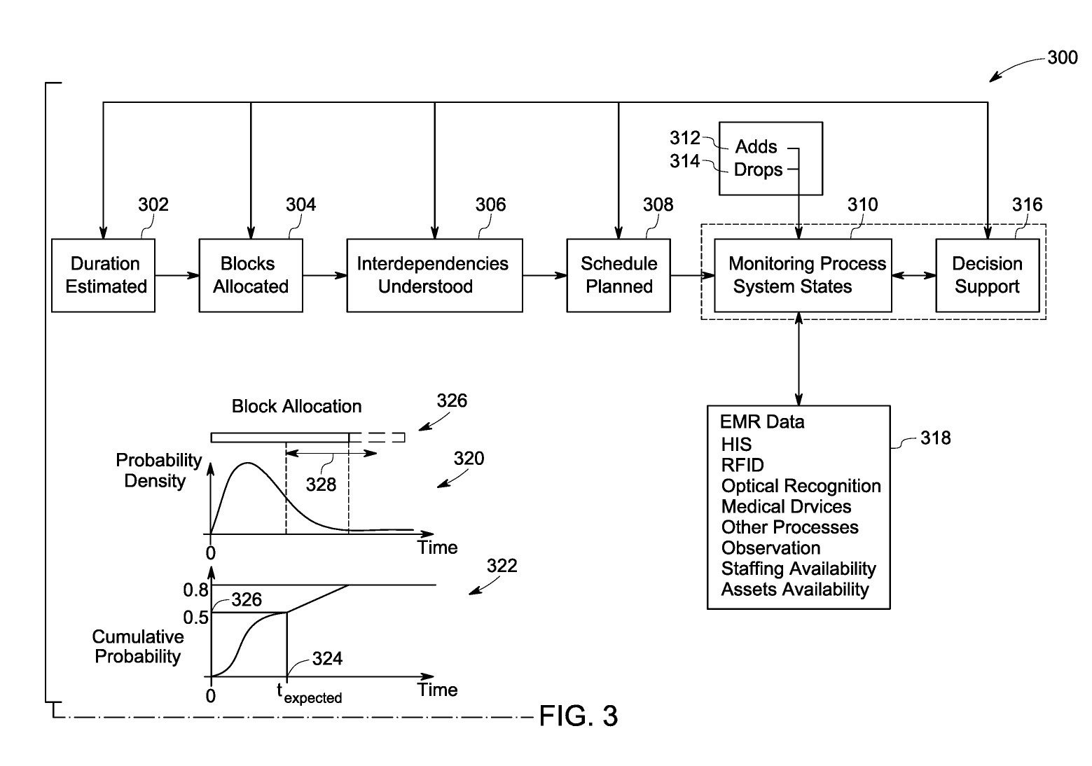

Certain exemplary embodiments describing automated systems and methods for reconfiguring patient care workflow in real-time based on patient state, alerting concerned caregivers and ensuring implementation of the rescheduled activities for optimizing the benefits of recuperative sleep to the patient will be discussed in greater detail with reference to FIGS. 2-3.

FIG. 2 illustrates an exemplary embodiment of the reasoning engine 200, such as the reasoning engine 116 of FIG. 1 for use in reconfiguring patient care workflow based on a phase of sleep of a patient. In certain embodiment, the reasoning engine 200 receives monitoring information from a plurality of sensors such as the sensors 106 and/or the information handling subsystem 104 of FIG. 1. As previously noted, the monitoring information, for example, may include physiological parameters of the subject, ambient environmental conditions and/or determined medical data.

In one embodiment, the reasoning engine 200 uses the monitoring information for estimating a phase of sleep of the patient. To that end, in one embodiment, the reasoning engine 200 may include a processing unit 202 configured to analyze the monitoring information for detecting information corresponding to patient motion. Alternatively, the processing unit 202 may estimate patient motion corresponding to limb movement, heartbeat and/or respiration. The processing unit 202 may then 116 compare the measured motion, heartbeat and respiration values with corresponding baseline information to detect the specific phase of sleep the patient is experiencing at a given time. If it is determined that the detected sleep phase is capable of providing significant recuperative benefits to the patient, the reasoning engine 200 may initiate an evaluation for reconfiguration of the scheduled patient care workflow.

To that end, the reasoning engine 200 may further include a duration estimator 204, scheduling workflow planner 206 and a user interface 208 configured to operate in concert for reconfiguring patient care workflows so as to optimize sleep quality of the patient. Particularly, these reasoning engine components may be configured to provide decisioning designed to enable rapid on-the-fly response to the detected sleep phase. Additionally, the these reasoning engine components may be configured to assess schedule risk, visualize likely process scenarios in advance, determine robust decisions in a dynamic environment for achieving operating objectives, learn from what transpired, achieve departmental objectives and/or include stakeholders in the clinical process.

To that end, in certain embodiments, the duration estimator 204 may be configured to characterize average duration times and/or variations from average duration times for a given procedure or activity for minimizing schedule risk. Further, the planner 206 may be configured to schedule procedures or activities in accordance with characterized times from the duration estimator 204. Particularly, the planner 206 may be configured to re-sort scheduled activities to alternative time slots so as to ensure availability of shared resources such as caregivers, medical devices, beds, rooms, time, capital and/or consumables in view of these resources' interdependencies to hospital operations and each other.

As previously noted, the planner 206 may be configured to optimize rescheduling of the resources so as to satisfy constraints and departmental objectives. In one example, the planner 206 may be configured to factor in preferences and/or availabilities, solve for the best departmental allocation of resources, assets, space and/or time, and through the allocation help achieve department policy objectives such as providing appropriate case mixes, outcomes, safety and/or incentives for desired behaviors. To that end, the planner 206 may include an optimizer 210 configured to optimize rescheduling of resources, assets and/or workflows using a multi-modality approach. Specifically, the optimizer 210 may perform the optimization so as to avoid under-utilization of precious patient care, resources and/or overscheduling resources leading to conflicts, delays in subsequent procedure starts and/or care provider burnout. Additionally, the planner 206 may provide the ability to simulate forward projection of resources, assets and/or workflows to test various alternative paths and/or contingencies that may be optimally selected by the planner 206.

In addition to configurable, total cost and service delivery objectives, the optimizer 210 has a measure of a patient's sleep quality. In certain embodiments, the sleep quality is typically a function of both uninterrupted duration. However, when interruptions must occur and when no other workflow or schedule combination is feasible, the optimizer 210 may infer the sleep quality using biometrical feedback. Such dynamic planning optimization by the optimizer 210 balances workflow objectives with sleep quality so as to find a global best satisfaction of objectives. A ratio of a utility function of departmental care delivery objectives divided by the recuperative state estimation of the patient's sleep, such as a Donald Ratio, may be used as a dynamic indicator for the optimized tradeoffs or for ongoing monitoring.

The planner 206 may employ the user interface 208 to allow a user to visualize variation determined by the duration estimator 204 and/or scheduling opportunities and constraints determined by the planner 206 for use in rescheduling procedures and activities. In certain embodiments, the user interface 208 may be a callable application as a subcomponent of a larger software system. Particularly, in one embodiment, the user interface 208 may be configured to indicate and display the determined variations along with suggestions as to “do-what” and enable “what-if” decisioning. The user interface 208, in another embodiment, may provide an overview of the schedule with other location and clinical information for alerting the staff about schedule deviations, corresponding causes and/or process interdependencies. In certain embodiments, the user interface 208 may also provide simulation results of alternative process paths, thus allowing constructive involvement of process stakeholders in the workflow reconfiguration.

An embodiment of the user interface 208 for use with the planner 206 will be referred to herein as “Day View” 212. FIG. 3 illustrates an exemplary implementation 300 of the Day View system such as the Day View 212 of FIG. 2 in the process context for use in the workflow reconfiguration process. In one embodiment, Day View 212 provides an interface for systems and methods for estimating a risk of not making the schedule and establishing corresponding alarm set points to minimize risks. To that end, at step 302, durations may be estimated from a historical book or record of business. If no historical data exists, data from other related facilities may be used. In certain embodiments, users may subscribe to services to receive or exchange data to aid in duration estimation 302 and other calculation. Additionally, access to other user data may allow comparison of procedure times between users/institutions. After duration estimation, at step 304, block allocation may occur. Further, interdependencies such as one x-ray machine needed in two rooms, people, surgeons and/or instruments needed in multiple places and/or times may be understood and planned into the schedule at steps 306 and 308, respectively.

(Day View) 212, implementing the functionality of the optimizer 210, may then allow monitoring of the patient care activities as the day progresses at step 310 in order to add (step 312), drop (step 314) and/or otherwise intervene in a prescribed schedule with automated adjustment and/or decision support at step 316. One or more designated analytical algorithms and other input 318, such as, electronic medical record (EMR) systems, healthcare information systems (HIS), status/monitoring systems, for example, RFID, patient call systems, patient bed monitoring, clinical systems, optical recognition for shape, data received from medical devices such as EKG system and anesthesia monitor, interaction with other processes, manual observations, staffing and/or equipment availability may be used for providing the decisioning support on the Day View 212.

Particularly, activity durations estimated at step 302 may be used to schedule time within available limits. In the embodiment illustrated in FIG. 3, the allocated blocks of time (from step 304) within which procedures may be booked for or by those entitled to provide the patient care activity may be defined. Further, a risk associated with the rescheduled activity, system level performance and/or optimization may be assessed, for example, using probability density functions (PDF) of time, such as illustrated in the graphical representation 320, for a given duration estimation of the patient care activity to be rescheduled. In one example, the PDF may be calculated from historical records of similar procedures. For example, the historical frequencies plotted in the histogram 320 may be normalized by one or more standard statistical techniques to create the PDF with area=1.

In certain embodiments, a schedule risk may also be calculated by integrating the PDF using statistical techniques to create a cumulative probability density function (CPDF) for duration of a specific activity, such as illustrated in the graphical representation 322, where a probability of the duration is made available. An expected time 324 to complete similar tasks may be found, for example, at the 50% probability 326, where at the expected time, it is 50% likely the procedure will finish sooner and 50% likely that it will take longer than this time value. A risk that a procedure will not finish within its allotted time may be determined by a probability provided by the CPDF 322 at the block of time allocated/expected 326 for the activity. Additionally, for one or more selected blocks of time allocated 328, the probability of the actual procedure completing at or before that time is found from the CPDF 322. Typically, the allocated block of time 326 may be used as a decision variable available to the scheduler and/or optimization algorithm used to achieve the schedule's objectives.

Certain embodiments facilitate dynamic, intelligent schedule change based on changes in the actual stochastic and interdependent processes of care occurring in the hospital balanced against sleep quality objectives for the patient. Such embodiments entail forecasting durations of procedures arranged within a schedule along with interdependencies of space, people, equipment, consumables and information (e.g., 302, 304, 306, 308). Actual process feedback may be provided such as from HIS, RFID, Optical recognition, telemetry and various clinical systems. An explicit mapping of interdependencies in process assets and their related task probabilistic durations of activities may be coupled to a simulation capability of the optimizer 210 (see FIG. 2) for finding feasible solutions.

Certain embodiments may employ a variety of simulation and forecast modalities for finding the feasible solutions for workflow reconfiguration. These modalities, for example, may include Critical path methods (CPM), forecast modalities, agent based simulation (AB), discrete event simulation (DE), continuous or system dynamic simulation (SD), and/or Monte Carlo simulation (MC) may be utilized. Particularly, in a presently contemplated embodiment, an extended form of CPM may be used to identify and organize interdependencies in a way that enables feasible solutions for workflow reconfiguration in view of one or more designated criteria. A first extension of the CPM is use of multi-modality simulation (for example, MC) to draw path-independent probabilities or duration into assumed task lengths.

Additionally, equipment and personnel availabilities may be incorporated as well. Alternatively, certain embodiments may employ a process model using discrete event-based, agent-based, and/or heuristic logic simulation or a historical pattern that models “what could be” and processes the model using, for example, Gantt/Pert based planner 206 with simulated and/or actual process activity feedback to aid in devising an efficient and robust reconfiguration of the patient care workflow.

With returning reference to FIG. 2, embodiments of the reasoning engine 200, thus, may address possible workflow reconfigurations with diagnoses capability in near real time, while providing decision support for prospective process recovery to either the originally prescribed state or a new one that optimizes sleep quality for the patient. To that end, in one embodiment, the reasoning engine 200 may be configured to simulate an impact of modifying the scheduled patient care workflow depending upon the detected sleep phase of the patient and availability and priority of assets, care providers, and other patients at one or more points in time. Such simulations may also aid in evaluating robustness of the reconfigured schedule to unplanned events and assessing schedule risk.

Accordingly, in one embodiment, the reasoning engine 200 provides decision support for determining a feasible and robust reconfiguration of the activities on the scheduled patient care workflow. Once the reasoning engine 200 determines the reconfiguration decisions, the patient care workflow and affected schedules may be updated to accommodate activities at alternative times. As previously noted, in certain embodiments, the updates are made with a view to enhance patient sleep and strengthen the patient care workflow against schedule risk, while also achieving one or more operating objectives or criteria established by the hospital or other healthcare facility.

In certain embodiments, the workflow reconfiguration decisions may be made based on historical data indicative of an effect of a particular activity and/or ambient condition on patient sleep and recovery. The reasoning engine 200, for example, may evaluate the historical data to identify sleep patterns in the presence and/or absence of disruptions caused by regularly scheduled activities, environmental stimuli and/or a pathological condition of the subject. Additionally, the reasoning engine 200 may also determine average time spent and the number and type of resources involved in discharging the scheduled activities over a period of time.

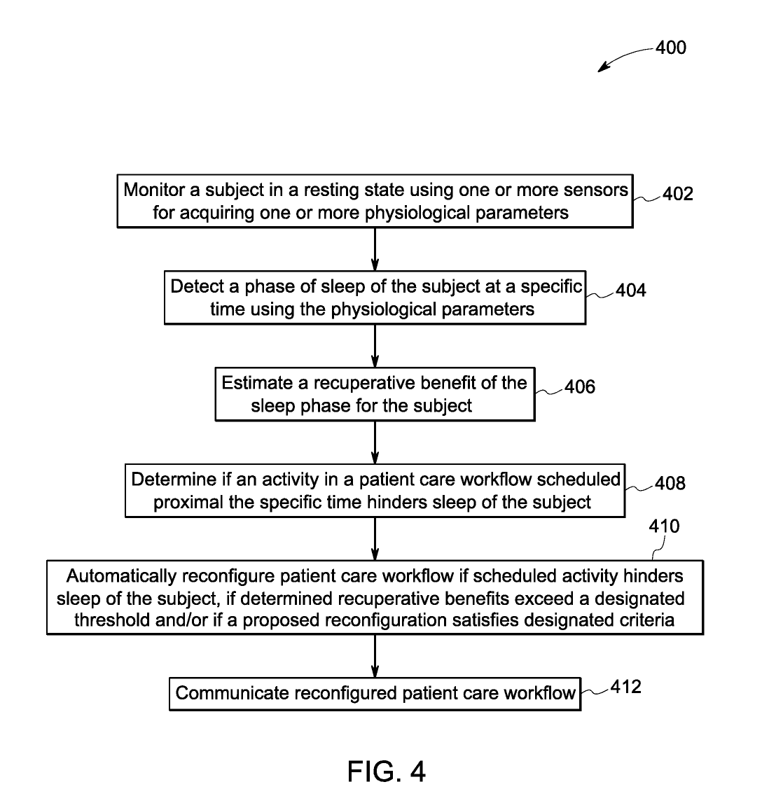

Alternatively, the reasoning engine 200 may implement a learning loop in a real-time and/or offline mode for identifying patterns showing a correlation between scheduled patient care activities and patient sleep and recovery. To that end the reasoning engine 200 may use techniques such as Artificial Neural Networks, Multivariate Regression, Analysis of Variance (ANOVA) and Correlation Analysis to refine predictive capability and tighten confidence bounds. Additionally new descriptive attributes may be appended in order to test and improve forecast accuracy for the identified patterns. The reasoning engine 200 may then use the identified patterns while making the workflow reconfiguration decisions in real-time. Certain exemplary embodiments of methods for reconfiguring patient care workflow in real-time based on a sleep phase of the patient will be described in greater detail with reference to FIG. 4.

FIG. 4 illustrates a flow chart 400 depicting an exemplary method for automated workflow management. The exemplary method may be described in a general context of computer executable instructions stored and/or executed on a computing system or a processor. Generally, computer executable instructions may include routines, programs, objects, components, data structures, procedures, modules, functions, and the like that perform particular functions or implement particular abstract data types. The exemplary method may also be practiced in a distributed computing environment where optimization functions are performed by remote processing devices that are linked through a wired and/or wireless communication network. In the distributed computing environment, the computer executable instructions may be located in both local and remote computer storage media, including memory storage devices.

Further, in FIG. 4, the exemplary method is illustrated as a collection of blocks in a logical flow chart, which represents operations that may be implemented in hardware, software, or combinations thereof. The various operations are depicted in the blocks to illustrate the functions that are performed, for example, during patient monitoring, data evaluation and workflow reconfiguration phases of the exemplary method. In the context of software, the blocks represent computer instructions that, when executed by one or more processing subsystems, perform the recited operations.

The order in which the exemplary method is described is not intended to be construed as a limitation, and any number of the described blocks may be combined in any order to implement the exemplary method disclosed herein, or an equivalent alternative method. Additionally, certain blocks may be deleted from the exemplary method or augmented by additional blocks with added functionality without departing from the spirit and scope of the subject matter described herein. For discussion purposes, the exemplary method will be described with reference to the elements of FIGS. 1-2.

Clinical procedures typically entail vast variations owing to a large amount of unplanned procedure time, emergency scenarios and unanticipated shortfall of medical resources. Despite the vast variation, in a clinical facility, patient care workflows are typically scheduled at a single prescribed time on a daily basis, such as at admission, during rounds and/or during shift planning. Additionally, aspects of the patient care workflow are often rigidly followed so as to maintain schedules of shared assets and care providers. Generally, these workflows are prescribed for servicing specific patient volumes based on historical patient care information and designated hospital protocols by attending physicians or hospital standing orders. However, in practice, patient care workflows end up being modified in real-time as variations in medical condition occur and the availability of resources such as doctors, nurses, rooms and equipment dynamically change, thus disrupting treatment plans related to the scheduled workflow.

The patient care workflow for a surgical patient, for example, may include activities such as physiotherapy, blood tests, medication, food, housekeeping, family visits and/or doctor consultation scheduled at different times during the day. In one example, the scheduled workflow may include physiotherapy during mornings and evenings, blood tests and medications to be administered every few hours, housekeeping at 10 am, lunch at noon, and doctor and/or family visits scheduled during the evenings. Additionally, ambient conditions such as light, sound such as from a telephone, vibrations and/or temperature in the patient's room may be kept constant or may be controlled based on the time of the day.

In conventional patient care facilities, such scheduled workflow related to the care protocols invoked for a patient's care plan is desired to be stringently followed yet is modified in real-time more as the routine than exception. Moreover, such on-the-fly modifications are often performed inefficiently due to, for example, availability of only partial information, ignorance about interdependencies and/or inability to appreciate the ramifications of rescheduling care activities and associated resources at a particular time instead of another time.

Certain conventional patient monitoring systems have endeavored to monitor the availability of resources for mitigating logistics during patient's day, a medical emergency and/or non-availability of medical resources. The cost and motivation for using conventional monitoring systems such as wearable devices, however, is often low. Moreover, even though these systems may gather useful data, the data often remains unorganized and/or merely informational in nature without providing reliable predictive, probabilistic or systemic capability for reconfiguring patient care workflows in real-time. Use of the unorganized data proves inadequate for mitigating unplanned processes in real-time, and thus, may result in poor reconfiguration choices, which in turn may lead to emotional decision making, delays, queues, overtime, caregiver burnout, low patient satisfaction, bottlenecked processes and/or missed or underperformed delivery of patient care.

Accordingly, embodiments of the present method allow for automated management of patient care workflows based on a detected resting state of the patient. To that end, at step 402, a subject in a resting state is monitored for acquiring one or more physiological parameters using one or more sensors. The sensors may include devices that may be attached to the patient via wires and/or electrodes, wireless wearable devices and/or devices that may provide non-invasive and contact-less monitoring of the patient.

Accordingly, in one embodiment, the sensors may include medical devices such as an ECG machine or a pulse oximeter configured to measure one or more physiological parameters of the subject continuously, periodically or at designated instants of time. In certain embodiments, the sensors, for example, include optical devices such as video optical sensing devices, range-controlled radars, RF/IR systems and/or thermal systems configured to non-invasively identify and monitor one or more ROI. Specifically, the sensors may be configured to monitor motion of the patient and of caregivers in the ROI. The motion of the caregivers within different regions of a patient room may be monitored for measuring adherence to patient care workflows such as following hand hygiene before and after patient contact, administering medication and tests, securing medical devices and/or recording measurements for various physiological and environmental parameters. Additionally, the sensors may also include devices configured to monitor ambient conditions, such as temperature, humidity, light exposure, sound levels and/or vibrations, which may affect patient sleep.

Particularly, in one example, a range-controlled Doppler radar may be employed for non-invasively monitoring physiological parameters of the patient disposed in a sleeping or resting state in a designated space even in the presence of obstructions. To that end, the range-controlled radar may be configured to transmit electromagnetic signals towards desired ROI in the patient room and sense corresponding echo signals reflected from the patient disposed in the resting state. The acquired information including the echo signals, in certain embodiments, may be communicated to an associated memory such as the memory device 112 of FIG. 1 for storage. Alternatively, the acquired information may be communicated to a processing unit such as the subsystem 104 of FIG. 1 for evaluation. Particularly, in one embodiment, the echo signals may be processed for extracting values corresponding to physiological parameters such as motion, heartbeat and respiration into signal frames based on their corresponding frequency band characteristics.

Further, at step 404, a phase of sleep of the subject may be detected using the extracted physiological parameters. Typically, rest is described in stages, or sleep cycles, each cycle characterized by a distinctive set of indicators that may be detected based on type and amplitude of brain waves measured by monitoring sensors, such as an electroencephalogram (EEG) machine. However, it may not be possible to procure contact measurements such as EEG signals for patients in every scenario. For example, in a home environment, an EEG machine may not be available. Accordingly, in such scenarios, non-contact sensors such as Doppler and/or optical devices may be used to infer the sleep phase using body movement and/or biometrical feedback measured, for example, using acquired video or radar signals. In certain embodiments, the sleep phase detection may also be inferred based upon prior study of other like patients or test sequences for subject patient so wired for a calibration period.

The sleep phases or cycles, generally include REM and NREM sleep, which in turn may be broken down into four further phases N1-N4, each of which may last from about 5-15 minutes. Typically, the N1 phase includes a transitional period between wakefulness and early sleep when the brain moves from alpha waves to theta waves. The N2 stage is characterized by EEG events known as “sleep spindles” and “k-complexes” in which the patient experiences brain wave bursts as the brain shuts down motor reflexes to prepare for deep rest. Accordingly, the patient experiences a decrease in heart rate and/or body temperature.

Further, N3 and N4 correspond to deep sleep phases, known as slow wave or delta sleep during which the body repairs itself, builds muscle and bone tissues, and strengthens the immune system. In a healthy person, the four stages of NREM sleep may be followed by REM sleep, followed by NREM sleep, and so on. REM sleep, accounting for up to 25 percent of total sleep in adults, is responsible for revitalizing the mind and is typically characterized by physiological parameters including rapid eye movement, muscular atonia and intense dreaming activity.

In one embodiment, the processing unit may determine patterns in the physiological parameters measured over a designated period of time. The determined patterns may then be compared with stored patterns corresponding to different phases of sleep to identify a match. Alternatively, one or more sensors such as an electroencephalogram (EEG) machine may be used for monitoring brain waves, eye movements and/or muscle tone of the patient for detecting the phase of sleep of the patient. By way of example, a monitoring system, such as the system 100 of FIG. 1 may detect if the patient is in NREM sleep or REM sleep at a specific time.

It may be noted that although the N1 and N2 phases of NREM sleep may be characterized by transitioning the body into a resting state, these phases may not provide significant recuperative benefits. The N3-N4 phases, however, may result in decreased blood pressure, slower and deeper breathing, and only slight brain activity. Particularly, in N3-N4 phases, the pituitary gland releases a shot of growth hormone that stimulates tissue growth and muscle repair. Accordingly, in N3-N4 phases, blood supply to muscles increases, in turn, delivering extra amounts of oxygen and nutrients to the muscles for facilitating healing and growth. N3 and N4 phases, thus, rejuvenate muscles and tissues, regenerate cells and expedite patient recovery.

Accordingly, at step 406, a recuperative benefit of the detected sleep phase may be estimated. The recuperative benefit, in certain embodiments, may be estimated using historical information that correlates the sleep phase with corresponding recuperative benefit imparted to patients. In certain other embodiments, however, the specific recuperative benefits imparted to the patient by the detected sleep phase may be estimated using previously determined medical information, a current pathological condition and/or a determined phase of recovery of the patient. Further, it may be determined if the estimated recuperative benefit exceeds a designated threshold. The designated threshold, for example, may depend upon significance of a determined patient care workflow and/or user input and may be defined using a determined value, range and/or percentage.

Additionally, at step 408, it may be determined if an activity in the patient care workflow scheduled proximal the specific time corresponding to detected sleep phase hinders sleep of the patient. To that end, a patient care workflow may be retrieved from a storage device or a central information handling system, such as the subsystem 104 of FIG. 1. In certain embodiments, this determination follows identification of the sleep phase of the patient. However, in other embodiments, the monitoring system, proximal a time at which a patient care activity is scheduled, may configure the sensors to monitor the patient and detect a sleeping phase. Subsequently, the monitoring system may determine if the scheduled activity will hinder patient sleep phase. In certain embodiments, the monitoring system may make this determination if the recuperative benefits of the detected sleep phase exceed the designated threshold.

Further, at step 410, the patient care workflow may be automatically reconfigured if the scheduled activity hinders patient sleep, the determined recuperative benefits exceed a designated threshold and proposed reconfiguration satisfies one or more designated criteria. The designated criteria, for example, may include a nature of the scheduled activity, projected availability of a resource associated with the scheduled activity, interdependencies associated with the resource, user input and/or determined patient care workflows. Further, the reconfiguration may be for a specific patient or an aggregated tradeoff at the department level for multiple patients.

In one embodiment, the patient care workflow may be reconfigured if the workflow includes an activity, rescheduling which may not affect the recovery of the patient. For example, if the patient care workflow includes housekeeping or physiotherapy scheduled at a time when the patient is in N3-N4 phase of sleep, a processing system such as the reasoning engine 116 of FIG. 1 may reconfigure the patient care workflow to reschedule housekeeping to an alternative time slot based on a projected availability of cleaning staff and/or other relevant resources at the rescheduled time. However, if the workflow includes a mandatory dose of a periodic medication, even if the patient is in N3-N4 sleep, no workflow reconfiguration may be initiated.

Further, in certain embodiments, the activity may be rescheduled so as to achieve designated objectives. The designated objectives, for example, may include improving sleep quality, optimal utilization of resources, reduced schedule risk, minimal impact on patient care workflows of one or more other patients and/or reduced operational costs. Particularly, in one embodiment, an activity may be rescheduled without disrupting workflows for one or more other patients, care providers and/or use of resources, for example, equipment, care providers and/or patient care area. In another embodiment, the automated workflow management system may propose a schedule of specific activities and corresponding interdependencies, while allowing a user to simulate several alternative plans and contingencies based on one or more of the designated criteria.

To that end, in one embodiment, the workflow management system may estimate duration of the scheduled activity, for example, from historical data and/or real-time monitoring. As previously noted, after duration estimation, the system may determine resource allocation for rearranging the activities on the patient care workflow. Additionally, task re-allocation may be performed to account for interdependencies such as availability of shared care providers, physical spaces, doctors and/or instruments in multiple places at designated times. In certain embodiments, the automated reconfiguration, for example, may be aided by input from associated healthcare systems, such as electronic medical record (EMR) systems, healthcare information systems (HIS), patient status/monitoring systems, optical recognition systems for recognizing shape of instrument and/or patient room activity, medical devices, manual observations, staffing and/or equipment management systems.

In certain further embodiments, the system may allow healthcare personnel to monitor patient sleep parameters and add, drop and/or otherwise intervene in a designated patient care workflow with reconfiguration and/or decision support. To that end, the system may be configured to provide a capability to view a single or multiple process metric, agent, and/or asset of the process as well as a dynamic impact on interdependencies. In one embodiment, for example, the system may provide a user scheduler interface that may allow for historical review such as replaying a day or past several days for determining process dynamics, training, and/or knowledge capture for use in future schedule configurations as well as for administrative activities such as determining costs, protocol verification and/or billing.

Additionally, resource utilization and consumption may be viewed to understand dynamic interdependencies between scheduling processes, identify assets and interdependencies that cause schedule variance, and to determine which schedule is likely to be met. In certain embodiments, a future schedule view may be provided to calculate “what-if” scenario testing to help understand schedule changes and effects of workflow reconfiguration, such as schedule additions or drops, resource availability, unforeseen delays and/or failures. Such future schedule extrapolation may allow for adherence to the designated criteria by managing variation and throughput, wait times, resource availability and/or utilization through careful workflow reconfiguration.

Certain embodiments may entail satisfying more than one criterion for implementing proposed workflow reconfiguration decisions. Additionally, certain criteria may be defined to be more significant than certain other criteria. Accordingly, in one embodiment, a designated weighted value may be assigned to one or more of such significant criteria while rescheduling an activity and reconfiguring the patient care workflow. To that end, the weight values may be assigned, for example, in view of a state of the patient, user input, caregiver's inputs, risk associated with rescheduling patient care activities, patient preference and/or the determined patient care workflows. For an aged patient and/or a patient recovering from an operation or rehabilitating an injury, reconfiguration of the patient care workflow may be favored if the patient is detected to be in N3-N4 NREM sleep. For mental illness, however, NREM sleep may not be as useful in rehabilitation as REM sleep. Accordingly, for a mentally ill patient, the workflow reconfiguration may be favored when the patient is detected to be in REM sleep.

In certain embodiments, the reasoning engine may be configured to reschedule one or more activities in the patient care workflow using a process model using discrete event-based, agent-based, and/or heuristic logic simulation that models “what could be” to a Gantt/Pert based scheduling engine with both simulated and actual process feedback. Alternatively, the reasoning engine may establish a learning loop configured to monitor the patient care in real-time and evaluate the acquired information over a period of time for identifying options that not only optimize recuperative sleep phases but also achieve departmental objectives.

Particularly, certain embodiments may propose a plurality of scheduling scenarios, for example, using a critical path method coupled to discrete event, agent, Monte Carlo and/or continuous simulation techniques. The scheduling scenarios may be manually input, dynamically reconfigured and/or simulated automatically to explore an available solution space and ramifications on current and future patient care activities to recommend reconfiguration options for the patient care workflow. As previously noted, the reconfigured patient care workflow may be determined so as to achieve one or more static, dynamic or criteria-dependent configurable objectives.

At step 412, the reconfigured patient care workflow may be communicated to one or more care providers and/or associated patient care systems. To that end, in certain embodiments, the system may generate, for example, an audio output and/or a visual output for notifying appropriate personnel and/or healthcare systems of one or more changes in the scheduled activities in a real-time and/or an offline mode. In one embodiment, the workflow reconfiguration in varied granularity such as per caregiver, department, role-type, shift, day, time period and/or room may be summarized and reported periodically. For example, information may be provided using a kiosk or workstation that may display and/or announce the reconfigured schedule, planning and/or decision support information. Alternatively, the system may sound an alarm, send a voicemail, text message and/or email to a mobile device of appropriate personnel and/or to another monitoring system through a wired and/or wireless link.

Embodiments of the present systems and methods, thus, describe an automated workflow management system for optimized patient care. Particularly, the system may be configured to reorganize patient care workflows in real-time to allow the patient to benefit from optimal recuperation derived from sleep. Particularly, in certain embodiments, the system may be configured to non-invasively detect a phase of sleep of the patient, determine a recuperative benefit of the detected sleep phase and identify any scheduled activities that may disrupt the sleep phase. The system may then compare the recuperative benefit with the logistics for rescheduling the patient care workflow and reschedule the workflow in real-time for optimizing the recuperative benefits of rehabilitating sleep phases.

Particularly, the automated reconfiguration of the patient care workflow may allow for optimal utilization of resources such as equipment, caregivers and/or physical spaces. Further, the automated system may allow for real-time control over ambient conditions and/or resource rescheduling for optimizing patient sleep. The optimized sleep aids in faster recovery, and it turn, lower costs. Faster recovery through the automated workflow reconfiguration may free resources for use by other patients, thus leading to additional revenue for hospitals and other healthcare facilities. Moreover, in certain embodiments, the reconfiguration decisions based on determined sleep patterns may be used for creating dashboards and/or designing future patient care workflows. An additional benefit of the reconfiguration may be improved patient experiences as a beneficial result of uninterrupted sleep and more restful sleep in cases when disruptive contact with the patient must occur.

It may be noted that although specific features of various embodiments of the present systems and methods may be shown in and/or described with respect to only certain drawings and not in others, this is for convenience only. It is to be understood that the described features, structures, and/or characteristics may be combined and/or used interchangeably in any suitable manner in the various embodiments, for example, to construct additional assemblies and techniques. Furthermore, the foregoing examples, demonstrations, and process steps, for example, those that may be performed by the information handling subsystem 104, the reasoning engine 116, reporting subsystem 120 and/or processing unit 202 may be implemented by a single device or a plurality of devices using suitable code on a processor-based system.

It should also be noted that different implementations of the present disclosure may perform some or all of the steps described herein in different orders or substantially concurrently, that is, in parallel. In addition, the functions may be implemented in a variety of programming languages, including but not limited to Python, C++ or Java. Such code may be stored or adapted for storage on one or more tangible, machine-readable media, such as on data repository chips, local or remote hard disks, optical disks (that is, CDs or DVDs), solid-state drives or other media, which may be accessed by a processor-based system to execute the stored code.

While only certain features of the present disclosure have been illustrated and described herein, many modifications and changes will occur to those skilled in the art. It is, therefore, to be understood that the appended claims are intended to cover all such modifications and changes as fall within the true spirit of the present disclosure.

1. A method, comprising:
acquiring, via one or more sensors, one or more physiological parameters of a patient in a resting state;
determining, via a processor, a current phase of sleep of the patient based at least in part on a comparison of the one or more physiological parameters to one or more respective baseline values;
estimating, via the processor, a recuperative benefit metric associated with the current phase of sleep of the patient;
determining, via the processor, whether the recuperative benefit metric associated with the current phase of sleep of the patient is greater than a benefit threshold;
identifying, via the processor, in response to determining that the recuperative benefit metric associated with the current phase of sleep of the patient is greater than the benefit threshold, one or more scheduled activities in a patient care workflow associated with the patient that may disrupt the current phase of sleep of the patient; and
automatically generating, via the processor, a reconfigured patient care workflow, in response to determining that one or more scheduled activities in the patient care workflow may disrupt the current phase of sleep of the patient.

2. The method of claim 1, wherein the one or more physiological parameters comprises patient movement, heart rate, respiration rate, blood pressure, body temperature, eye movement, muscle tone, brain wave activity, or a combination thereof.

3. The method of claim 2, wherein determining the current phase of sleep of the patient comprises determining whether the current phase of sleep of the patient corresponds to an N3 or N4 phase of sleep.

4. The method of claim 2, wherein the one or more respective baseline values correspond to decreased blood pressure, decreased respiration rate, only slight brain activity, or a combination thereof.

5. The method of claim 1, wherein automatically generating the reconfigured patient care workflow comprises rescheduling, via the processor, the one or more scheduled activities in the patient care workflow that may disrupt the current phase of sleep of the patient.

6. The method of claim 5, wherein automatically generating the reconfigured patient care workflow comprises:
determining, via the processor, whether rescheduling the one or more scheduled activities in the patient care workflow that may disrupt the current phase of sleep of the patient may affect recovery of the patient; and
automatically generating, via the processor, the reconfigured patient care workflow, in response to determining that rescheduling the one or more scheduled activities in the patient care workflow that may disrupt the current phase of sleep of the patient will not affect recovery of the patient.

7. The method of claim 6, wherein rescheduling the one or more scheduled activities in the patient care workflow that may disrupt the current phase of sleep of the patient is based at least in part on one or more designated criteria, wherein the designated criteria comprise a nature of the one or more scheduled activities, projected availability of one or more resources associated with the one or more scheduled activities, interdependencies associated with the one or more resources, user input, other determined patient care workflows, or a combination thereof.

8. The method of claim 1, wherein estimating the recuperative benefit metric associated with the current phase of sleep of the patient is based at least in part on historical information that correlates the phase of sleep with corresponding recuperative benefits, previously determined medical information associated with the patient, a pathological condition associated with the patient, a phase of recovery of the patient, or a combination thereof.

9. The method of claim 1, comprising communicating, via the processor, the reconfigured patient care workflow to one or more care providers or patient care systems associated with the patient care workflow of the patient.

10. The method of claim 1, wherein automatically generating the reconfigured patient care workflow comprises automatically generating the reconfigured patient care workflow in real-time.

11. A system, comprising:
one or more sensors configured to non-invasively acquire one or more physiological parameters of a patient in a resting state; and
a processor communicatively coupled to the one or more sensors, wherein the processor is configured to:
determine a current phase of sleep of the patient based at least in part on the one or more physiological parameters;
estimate a recuperative benefit metric associated with the current phase of sleep of the patient;
determine whether the recuperative benefit metric associated with the current phase of sleep of the patient is greater than a benefit threshold;
identify, in response to determining that the recuperative benefit metric associated with the current phase of sleep of the patient is greater than the benefit threshold, one or more scheduled activities in a patient care workflow associated with the patient that may disrupt the current phase of sleep of the patient; and
generate, in response to determining that one or more scheduled activities in the patient care workflow may disrupt the current phase of sleep of the patient, a reconfigured patient care workflow in real-time.

12. The system of claim 11, wherein the one or more physiological parameters comprises patient movement, heart rate, respiration rate, blood pressure, body temperature, eye movement, muscle tone, brain wave activity, or a combination thereof.

13. The system of claim 11, wherein the processor is configured to determine whether the current phase of sleep of the patient corresponds to an N3 or N4 phase of sleep.

14. The system of claim 11, wherein the processor is configured to determine the current phase of sleep of the patient based at least in part on a comparison of the one or more physiological parameters to one or more respective baseline values or ranges.

15. The system of claim 12, wherein the one or more respective baseline values correspond to decreased blood pressure, decreased respiration rate, only slight brain activity, or a combination thereof.

16. The system of claim 11, wherein the processor is configured to:
determine one or more patterns present in the acquired one or more physiological parameters;
compare the determined one or more patterns in the acquired one or more physiological parameters with stored known patterns corresponding to different phases of sleep; and
determine the current phase of sleep of the patient based at least in part on the comparison of the determined one or more patterns in the acquired one or more physiological parameters with the stored known patterns.

17. The system of claim 11, wherein the one or more sensors comprise one or more range-controlled radars configured to transmit a radar signal and receive a reflected radar signal for acquiring the one or more physiological parameters corresponding to the patient in a resting state.

18. The system of claim 11, wherein the processor is configured to:
determine whether rescheduling the one or more scheduled activities in the patient care workflow that may disrupt the current phase of sleep of the patient may affect recovery of the patient; and
automatically generate, in response to determining that rescheduling the one or more scheduled activities in the patient care workflow that may disrupt the current phase of sleep of the patient will not affect recovery of the patient, the reconfigured patient care workflow in real-time.

19. The system of claim 11, wherein the processor is configured to communicate the reconfigured patient care workflow to one or more care providers or patient care systems associated with the patient care workflow of the patient.

20. A non-transitory computer-readable medium comprising computer-executable instructions configured to cause at least one processor to:
determine a current phase of sleep of the patient based at least in part on a comparison of the one or more physiological parameters to one or more respective baseline values or ranges;
estimate a recuperative benefit metric associated with the current phase of sleep of the patient;
determine whether the recuperative benefit metric associated with the current phase of sleep of the patient is greater than a benefit threshold;
identify, in response to determining that the recuperative benefit metric associated with the current phase of sleep of the patient is greater than the benefit threshold, one or more scheduled activities in a patient care workflow associated with the patient that may disrupt the current phase of sleep of the patient;
automatically generate a reconfigured patient care workflow in real-time, in response to determining that one or more scheduled activities in the patient care workflow may disrupt the current phase of sleep of the patient, wherein the reconfigured patient care workflow comprises one or more rescheduled activities corresponding to the one or more scheduled activities in a patient care workflow associated with the patient that may disrupt the current phase of sleep of the patient; and
communicate the reconfigured patient care workflow to one or more care providers or patient care systems associated with the patient care workflow of the patient.
